(19) United States
(12) Patent Application Publication
(10) Pub. No.: US 2017/0183638 A1
(43) Pub. Date: Jun. 29, 2017

(54) SOLUBLE METHANE MONOOXYGENASE PROTEIN VARIANT AND METHOD OF REDUCING CONCENTRATION OF FLUORINATED METHANE IN SAMPLE USING THE SAME

(71) Applicant: Samsung Electronics Co., Ltd., Suwon-si (KR)

(72) Inventors: Yukyung Jung, Hwaseong-si (KR); Dongsik Yang, Seoul (KR); Jinhwan Park, Suwon-si (KR); Taeyong Kim, Daejeon (KR); Anirban Bhaduri, Bangalore (IN); Tadi Venkata Siva Kumar, Bangalore (IN)

(21) Appl. No.: 15/389,571

(22) Filed: Dec. 23, 2016

(30) Foreign Application Priority Data
Dec. 23, 2015 (KR) 10-2015-0185093
Jun. 17, 2016 (KR) 10-2016-0075832
Aug. 26, 2016 (KR) 10-2016-0109545

(51) Int. Cl. C12N 9/02; B01D 53/84; C12N 15/70

(52) U.S. Cl. CPC C12N 9/0073; C12Y 114/13025; C12N 15/70; B01D 53/84; B01D 2255/804; B01D 2251/95

(57) ABSTRACT

Provided are a recombinant microorganism including an exogenous gene encoding a soluble methane monooxygenase protein, a composition including the soluble methane monooxygenase, which is used for removing CHₙF₄₋ₙ (n is an integer of 0 to 3) in a sample, and a method of reducing a concentration of CHₙF₄₋ₙ in the sample.

### CROSS-REFERENCE TO RELATED APPLICATIONS

This application claims the benefit of Korean Patent Application No. 10-2015-0185093, filed on Dec. 23, 2015, Korean Patent Application No. 10-2016-0075832, filed on Jun. 17, 2016, and Korean Patent Application No. 10-2016-0109545, filed on Aug. 26, 2016, in the Korean Intellectual Property Office, the disclosures of which are incorporated herein in their entireties by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 52,893 Byte ASCII (Text) file named “727295_ST25.TXT,” created on Dec. 20, 2016.

### BACKGROUND

1. Field

The present disclosure relates to a recombinant microorganism comprising an exogenous gene encoding a soluble methane monooxygenase protein variant, a composition including the soluble methane monooxygenase protein, which is used for removing CHₙF₄₋ₙ (n is an integer of 0 to 3) in a sample, and a method of reducing a concentration of CHₙF₄₋ₙ in the sample using the protein.

2. Description of the Related Art

The emission of greenhouse gases, which have accelerated global warming, is a serious environmental problem, and regulations to reduce and prevent the emission of greenhouse gases have been tightened. Among the greenhouse gases, fluorinated gases (F-gas) such as perfluorocarbons (PFCs), hydrofluorocarbons (HFCs), and sulfur hexafluoride (SF₆) show low absolute emission, but have a long half-life and a very high global warming potential, resulting in a significant adverse environmental impact. The amount of F-gas emitted from the semiconductor and electronics industries have exceeded the assigned amount of greenhouse gas emissions and continues to increase. Therefore, the costs required for degradation of greenhouse gases and greenhouse gas emission allowances are increasing every year.

A pyrolysis or catalytic thermal oxidation process has been generally used in the decomposition of F-gas. However, this process has the disadvantages of limited decomposition rate, emission of secondary pollutants, and high cost. To solve this problem, biological decomposition of F-gas using a microbial biocatalyst has been adopted, and it is expected to overcome the limitations of the chemical decomposition process and to treat F-gas in more economical and environmentally-friendly manner.

Therefore, there is a need to identify new microbial biocatalysts and methods that can economically and efficiently decompose F-gas. This invention provides such a microorganism and method.

### SUMMARY

An aspect provides a recombinant microorganism comprising an exogenous gene encoding a soluble methane monooxygenase protein or a variant thereof.

Another aspect provides a composition comprising the soluble methane monooxygenase protein or the variant thereof, which is used for removing fluorinated methane represented by CHₙF₄₋ₙ (n is an integer of 0 to 3) in a sample.

Still another aspect provides a method of reducing the concentration of fluorinated methane in a sample, the method comprising contacting the soluble methane monooxygenase protein or variant thereof with the sample containing fluorinated methane to reduce the concentration of fluorinated methane in the sample.

Still another aspect provides a variant of the soluble methane monooxygenase protein and a polynucleotide encoding the same.

### BRIEF DESCRIPTION OF THE DRAWINGS

These and/or other aspects will become apparent and more readily appreciated from the following description of the embodiments, taken in conjunction with the accompanying drawings in which:

FIG. 1A shows a vector map of a pET28a-mmoXYBZDC vector;

FIG. 1B shows a vector map of a pETDuet-mmoXY-ZD vector;

FIG. 1C shows a vector map of a pACYCDuet-mmoBC vector;

FIG. 1D shows a vector map of a pACYCDuet-mmoG-BC vector;

FIG. 2 shows changes in a headspace concentration of CHF₃ when recombinant E. coli was cultured in a medium contacted with CHF₃-containing gas;

FIG. 3 shows changes in a headspace concentration of CHCl₃ when recombinant E. coli was cultured in a CHCl₃-containing medium;

FIG. 4 shows changes in a headspace concentration of CF₄ when recombinant E. coli was cultured in a medium contacted with CF₄-containing gas

FIG. 5 shows changes in a headspace concentration of CF₄ over time when recombinant E. coli BL21/pET28a-mmoXYBZDC was cultured for 7 days in a medium contacted with CF₄-containing gas.

### DETAILED DESCRIPTION

The term “gene”, as used herein, refers to a nucleic acid fragment that expresses a specific protein, and may include a coding region or a non-coding regulatory region (e.g., 5′-non-coding sequence and/or a 3′-non-coding sequence). The regulatory region may include a promoter, an enhancer, an operator, a ribosome binding site, a polyA binding sequence, a terminator region, etc.

A “sequence identity” of a nucleic acid or a polypeptide, as used herein, refers to the extent of identity between two or more nucleotide or amino acid sequences obtained after the sequences are aligned so as to best match. A percentage of sequence identity may be calculated by, for example, comparing two optimally aligned sequences, determining the number of locations in which the same amino acids or nucleic acids appear to obtain the number of matching locations, dividing the number of matching locations by the total number of locations in the comparable regions (e.g., the size of a range), and multiplying a result of the division by 100 to obtain the percentage of the sequence identity. The percentage of the sequence identity may be determined using a known sequence comparison program, for example, BLASTN or BLASTP (NCBI), CLC Main Workbench (CLC bio), MegAlign™ (DNASTAR Inc), etc. Unless otherwise specified, selection of parameters used for operating the program is as follows: Ktuple=2, Gap Penalty=4, and Gap length penalty=12. In this regard, a range included in the “corresponding” sequence may be a range of E-value 0.00001 and H-value 0.001.

In confirming many different polypeptides or polynucleotides having the same or similar function or activity, sequence identities at several levels may be used. For example, the sequence identities may include about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, about 99% or higher, or about 100%.

An aspect provides a recombinant microorganism comprising an exogenous gene encoding a soluble methane monooxygenase (sMMO) protein or a variant thereof.

Soluble methane monooxygenase (sMMO) is an oxidoreductase enzyme, that contains three components of a hydroxylase, a reductase, and a regulatory component. The hydroxylase component exists in a dimeric form, namely, (aβv)2. The structure of a hydroxylase component monomer consists of MmoX, MmoY, and MmoZ. The reductase component MmoC contains a prosthetic group and oxidizes NADH to NAD⁺. The regulatory component MmoB is involved in electron transfer from the reductase component to the hydroxylase component. MmoD is also a component of sMMO, but its components or functions have not been clearly revealed.

With regard to the recombinant microorganism, the sMMO protein may belong to EC 1.14.13.25. The sMMO protein may be derived from Methylococcus capsulatus (Bath). The sMMO protein may include MmoX or a variant thereof, MmoY, MmoZ, MmoB, MmoC, and MmoD. MmoD may be also called OrfY. MmoX, MmoY, MmoZ, MmoB, MmoC, and MmoD may have 95% or higher sequence identity to amino acid sequences of SEQ ID NOS: 1, 3, 5, 7, 9, and 11, respectively. Polynucleotides encoding MmoX, MmoY, MmoZ, MmoB, MmoC, and MmoD may have nucleotide sequences of SEQ ID NOS: 2, 4, 6, 8, 10, and 12, respectively. With regard to the recombinant microorganism, the gene may include a polynucleotide having the nucleotide sequence of SEQ ID NO: 2 or a polynucleotide encoding the variant of MmoX, a polynucleotide having the nucleotide sequence of SEQ ID NO: 4, a polynucleotide having the nucleotide sequence of SEQ ID NO: 6, a polynucleotide having the nucleotide sequence of SEQ ID NO: 8, a polynucleotide having the nucleotide sequence of SEQ ID NO: 10, and a polynucleotide having the nucleotide sequence of SEQ ID NO: 12. With regard to the recombinant microorganism, the gene encoding the sMMO may comprise the nucleotide sequence of SEQ ID NO: 31.

The variant may have an amino acid alteration at the amino acid residue corresponding to position T213 in the MmoX amino acid sequence of SEQ ID NO: 1. The MmoX or the variant thereof may have an activity belonging to EC 1.14.13.25. Holoenzyme including MmoX or variant thereof may have an activity belonging to EC 1.14.13.25. The MmoX variant may comprise a replacement of the threonine at position 213 SEQ ID NO: 1 with any other amino acids, for example, 19 natural amino acids. The MmoX variant may comprise a replacement of the threonine at position 213 with tyrosine (Y), serine (S), lysine (K), histidine (H), or glutamic acid (E) (i.e., T213Y, T213S, T213K, T213H, or T213E) A gene encoding the MmoX variant may be a gene encoding the amino acid sequence of SEQ ID NO: 1 comprising T213Y, T213S, T213K, T213H, or T213E. The variant gene may have a nucleotide sequence of SEQ ID NO: 42, 43, 44, 45, or 46.

The enzyme belonging to EC 1.14.13.25 may catalyze the following reaction:

Methane+NAD(P)H+H⁺+O₂ [symbol] Methanol+NAD(P)⁺+H₂O

The recombinant microorganism may further comprise an exogenous gene encoding MmoG. MmoG may have 95% or higher sequence identity to an amino acid sequence of SEQ ID NO: 13. A polynucleotide encoding MmoG may have a nucleotide sequence of SEQ ID NO: 14.

As used herein, the term “corresponding” refers to the amino acid position of a protein of interest that aligns with the mentioned position (e.g., position T213 of SEQ ID NO: 1) of a reference protein when amino acid sequences (e.g., SEQ ID NO:1) of the protein of interest and the reference protein are aligned using an art-acceptable protein alignment program (e.g., BLAST pairwise alignment or Lipman-Pearson Protein Alignment program) with the following parameters: Ktuple=2, Gap Penalty=4, and Gap length penalty=12. Database (DB) storing the reference sequence may be RefSeq non-redundant proteins of NCBI. In this regard, a range included in the “corresponding” sequence may be a range of E-value 0.00001 and H-value 0.001.

Examples of the proteins (hereinafter, referred to as “homologs of MmoX”) having the amino acid residue corresponding to the position T213 of the amino acid sequence of SEQ ID NO: 1, obtained according to the alignment conditions, are as in the following Table 1.

TABLE 1

| NO. | NCBI ID |
|---|---|
| 1 | gi\|13399575 |
| 2 | gi\|586941001 |
| 3 | gi\|697077732 |
| 4 | gi\|89572582\| |
| 5 | gi\|501586003 |
| 6 | gi\|519018694 |
| 7 | gi\|764628036 |
| 8 | gi\|501360567 |
| 9 | gi\|640365325 |
| 10 | gi\|88656498 |
| 11 | gi\|70671762 |
| 12 | gi\|70671680 |
| 13 | gi\|21239746 |
| 14 | gi\|41019259 |
| 15 | gi\|269980461 |
| 16 | gi\|727259737 |
| 17 | gi\|503977368 |
| 18 | gi\|820791923 |
| 19 | gi\|115511382 |
| 20 | gi\|397782080 |
| 21 | gi\|806818575 |
| 22 | gi\|494004786 |
| 23 | gi\|504618264 |

The recombinant microorganism may be bacteria or fungi. The bacteria may be Gram-positive or Gram-negative bacteria. The Gram-negative bacteria may belong to the family Enterobacteriaceae. The Gram-negative bacteria may belong to the genus Escherichia, the genus Samonella, the genus Xanthomonas, or the genus Pseudomonas. The genus Escherichia microorganism may be E. coli. The genus Xanthomonas microorganism may include Xanthobacter autotrophicus. Gram-positive bacteria may belong to the genus Corynebacterium or the genus Bacillus. The recombinant microorganism may be introduced with a polynucleotide having a nucleotide sequence of SEQ ID NO: 31.

Another aspect provides a composition comprising the soluble methane monooxygenase (sMMO) protein or the variant thereof, which is used for removing fluorinated methane represented by CHₙF₄₋ₙ (n is an integer of 0 to 3) in a sample. Unless otherwise specified, the recombinant sMMO protein or the variant thereof is the same as described above.

With regard to the composition, the fluorinated methane may be, for example, CHF₃, CH₂F₂, CH₃F, or CF₄. The term “removing” includes reducing the concentration of fluorinated methane in the sample. Reducing includes complete removal.

With regard to the composition, the sMMO protein may belong to EC 1.14.13.25. The sMMO protein may be derived from Methylococcus capsulatus (Bath). The sMMO protein may include MmoX or a variant thereof, MmoY, MmoZ, MmoB, MmoC, and MmoD. MmoX or the variant thereof, MmoY, MmoZ, MmoB, MmoC, and MmoD may have 95% or higher sequence identity to amino acid sequences of SEQ ID NOS: 1, 3, 5, 7, 9, and 11, respectively. Polynucleotides encoding MmoX or the variant thereof, MmoY, MmoZ, MmoB, MmoC, and MmoD are the same as described above.

With regard to the composition, the sMMO protein may be expressed from the recombinant microorganism including the exogenous gene encoding the protein. The composition may include the recombinant microorganism, a lysate thereof, or a water-soluble material fraction of the lysate. The exogenous gene included in the recombinant microorganism may comprise a polynucleotide having the nucleotide sequence of SEQ ID NO: 2 or a polynucleotide encoding the variant of MmoX, a polynucleotide having the nucleotide sequence of SEQ ID NO: 4, a polynucleotide having the nucleotide sequence of SEQ ID NO: 6, a polynucleotide having the nucleotide sequence of SEQ ID NO: 8, a polynucleotide having the nucleotide sequence of SEQ ID NO: 10, and a polynucleotide having the nucleotide sequence of SEQ ID NO: 12. The exogenous gene encoding the sMMO in the recombinant microorganism may comprise the nucleotide sequence of SEQ ID NO: 31. The recombinant microorganism may be introduced with the polynucleotide having the nucleotide sequence of SEQ ID NO: 31.

The recombinant microorganism may be bacteria or fungi. The bacteria may be Gram-positive or Gram-negative bacteria. The Gram-negative bacteria may belong to the family Enterobacteriaceae. The Gram-negative bacteria may belong to the genus Escherichia, the genus Samonella, the genus Xanthomonas, or the genus Pseudomonas. The genus Escherichia microorganism may be E. coli. The genus Xanthomonas microorganism may include Xanthobacter autotrophicus. Gram-positive bacteria may belong to the genus Corynebacterium or the genus Bacillus.

With regard to the composition, the removing of fluorinated methane may include cleaving of C—F bonds of fluorinated methane, converting of fluorinated methane into other materials, or reducing of the concentration of fluorinated methane by intracellular accumulation. The converting may be introducing of a hydrophilic group such as a hydroxyl group into fluorinated methane or introducing of a carbon-carbon double bond or a carbon-carbon triple bond thereto.

With regard to the composition, the sample may be in a liquid or gas state. The sample may be industrial waste water or waste gas.

Further, the composition may be used for removing ethyl acetate.

Still another aspect provides a method of reducing the concentration of fluorinated methane in a sample, the method comprising contacting the soluble methane monooxygenase (sMMO) protein or the variant thereof with the sample containing fluorinated methane represented by CHₙF₄₋ₙ (n is an integer of 0 to 3) to reduce the concentration of fluorinated methane in the sample. Unless otherwise specified, the recombinant sMMO protein or the variant thereof is the same as described above.

The contacting may be gas-liquid contact of contacting a gas sample with a liquid containing the sMMO protein or the variant thereof. Further, the contacting may be liquid-liquid contact of contacting a liquid sample with a liquid containing the sMMO protein or the variant thereof. The liquid-liquid contact includes mixing thereof.

With regard to the method, the sMMO protein may be expressed from the recombinant microorganism including the exogenous gene encoding the protein. The composition may include the recombinant microorganism, a lysate thereof, or a water-soluble material fraction of the lysate. The exogenous gene included in the recombinant microorganism is the same as described above.

The recombinant microorganism may be bacteria or fungi. The bacteria may be Gram-positive or Gram-negative bacteria. The Gram-negative bacteria may belong to the family Enterobacteriaceae. The Gram-negative bacteria may belong to the genus Escherichia, the genus Samonella, the genus Xanthomonas, or the genus Pseudomonas. The genus Escherichia microorganism may be E. coli. The genus Xanthomonas microorganism may include Xanthobacter autotrophicus. Gram-positive bacteria may belong to the genus Corynebacterium or the genus Bacillus.

With regard to the method, the contacting may be performed in the sealed container under conditions where the recombinant microorganism may survive or be viable. The phrase “sealed container” represents an air tight condition. The conditions where the recombinant microorganism may survive or be viable may be conditions where the recombinant microorganism may be allowed to proliferate or to be in a resting state. In this case, the contacting may be culturing of the microorganism in the presence of fluorinated methane. The culturing may be performed under aerobic or anaerobic conditions.

With regard to the method, the sample may be in a liquid or gas state. The sample may be industrial waste water or waste gas.

Further, the method may be used for removing ethyl acetate.

Still another aspect provides the MmoX variant having an amino acid alteration at an amino acid residue corresponding to the position T213 in MmoX of the amino acid sequence of SEQ ID NO: 1, and having an activity belonging to EC 1.14.13.25. The variant may have replacement of the amino acid residue corresponding to the position T213 with Y, S, K, H, or E in MmoX having the amino acid sequence of SEQ ID NO: 1. The amino acid alteration may be replacement of T213Y, T213S, T213K, T213H, or T213E in MmoX having the amino acid sequence of SEQ ID NO: 1.

Still another aspect provides a polynucleotide encoding the MmoX variant having an amino acid alteration at an amino acid residue corresponding to the position T213 in MmoX of the amino acid sequence of SEQ ID NO: 1 and having an activity belonging to EC 1.14.13.25.

The variant may have replacement of the amino acid residue corresponding to the position T213 with Y, S, K, H, or E in MmoX having the amino acid sequence of SEQ ID NO: 1. The amino acid alteration may be replacement of T213Y, T213S, T213K, T213H, or T213E in MmoX having the amino acid sequence of SEQ ID NO: 1. The polynucleotide encoding the MmoX variant may encode the variant having replacement of the amino acid residue corresponding to the position T213 with Y, S, K, H, or E in MmoX having the amino acid sequence of SEQ ID NO: 1. The polynucleotide encoding the MmoX variant may be a gene encoding the variant having replacement of T213Y, T213S, T213K, T213H, or T213E in MmoX having the amino acid sequence of SEQ ID NO: 1. The gene may have a nucleotide sequence of SEQ ID NO: 42, 43, 44, 45, or 56.

The polynucleotide encoding the sMMO protein or variant thereof may be included in a vector. The vector may be any vector, as long as it is used to introduce the polynucleotide into microorganisms. The vector may be a plasmid or viral vector.

The recombinant microorganism according to an aspect may be used for removing fluorinated methane in the sample.

The method of reducing the concentration of fluorinated methane in the sample according to still another aspect may efficiently reduce the concentration of fluorinated methane in the sample.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

Hereinafter, the present invention will be described in more detail with reference to Examples. However, these Examples are for illustrative purposes only, and the scope of the present invention is not intended to be limited by these Examples.

### Example 1: Recombinant E. coli Expressing sMMO Gene and Removal of Halomethane in Sample by Using the Same

In this Example, a recombinant E. coli expressing an sMMO gene was prepared, and its ability to remove halomethane (i.e., CHF₃ or CHCl₃) in a sample was examined.

(1) Preparation of Recombinant E. coli Expressing sMMO Gene

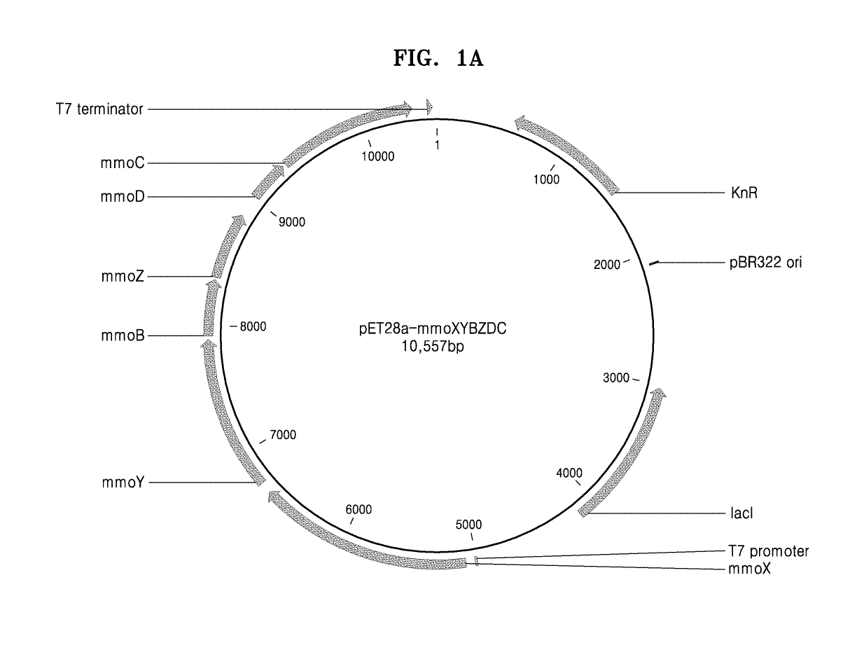

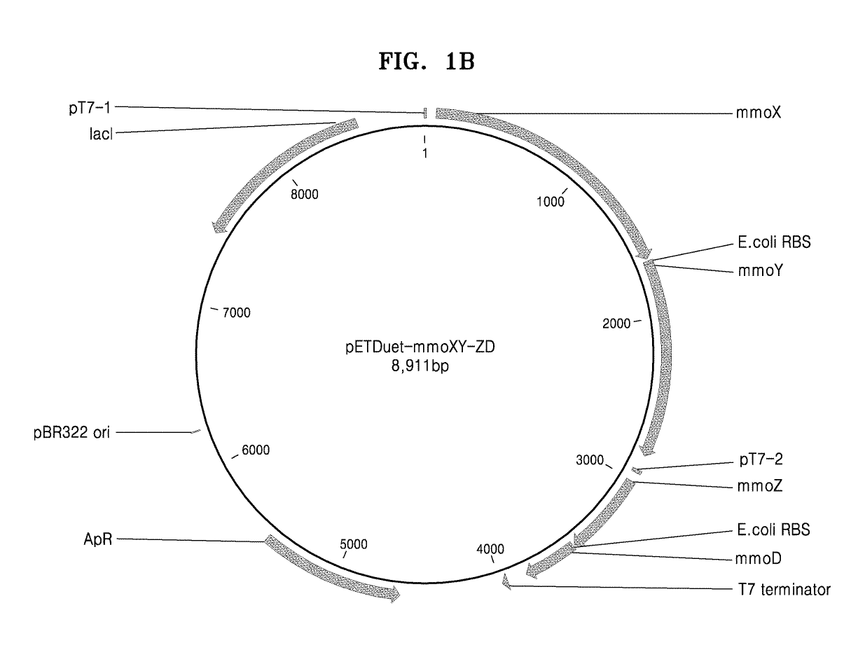

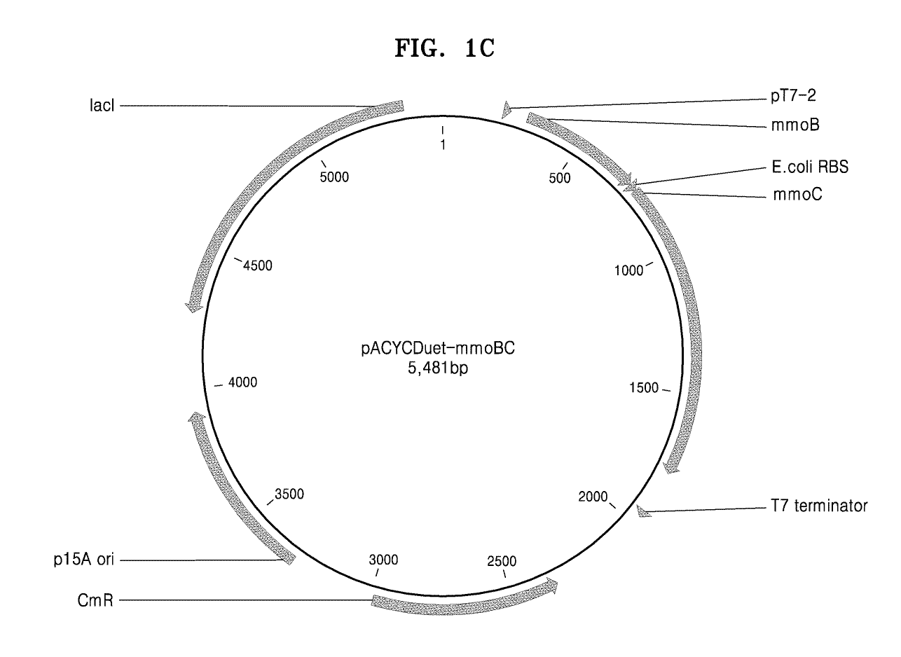

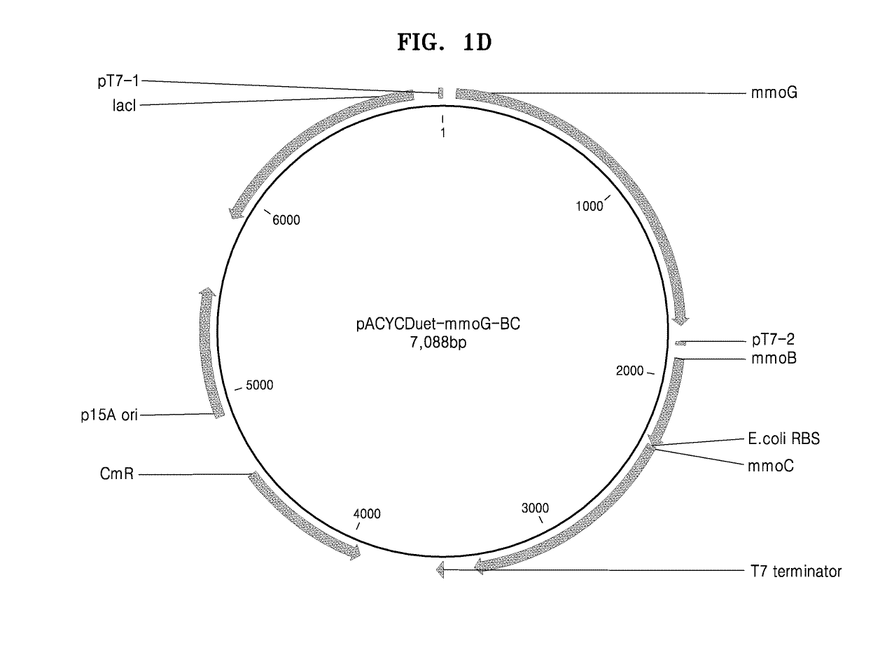

sMMO genes, i.e., mmoX, mmoY, mmoZ, mmoB, mmoC, mmoD, and mmoG genes were amplified from Methy/ococcus capsu/atus (Bath) strain. mmoX, mmoY, mmoZ, mmoB, mmoC, mmoD, and mmoG genes have nucleotide sequences of SEQ ID NOS: 2, 4, 6, 8, 10, 12, and 14, and they encode amino acid sequences of SEQ ID NO: 1, 3, 5, 7, 9, 11, and 13, respectively. In detail, PCR was performed using chromosomal DNA of Methylococcus capsulatus (Bath) strain (ATCC 33009D-5) as a template and a set of primers having nucleotide sequences of SEQ ID NOS: 15 and 16 were used to amplify a region of SEQ ID NO: 31 including all of the mmoX, mmoY, mmoZ, mmoB, mmoC, and mmoD genes. An amplified gene fragment was ligated with pET28a (Novagen, Cat. No. 69864-3) digested with restriction enzymes, NcoI and XhoI, using an InFusion Cloning Kit (Clontech Laboratories, Inc.) to prepare a pET28a-mmoXYBZDC vector. FIG. 1A shows a vector map of the pET28a-mmoXYBZDC vector.

Further, to express the sMMO gene using E. coli ribosome binding site (RBS), mmoX, mmoY, mmoZ, mmoB, mmoC, and mmoD were amplified, and then inserted into an expression vector. A region including the mmoX and mmoY genes was amplified using an mmoX gene fragment which was amplified by PCR using a set of primers of nucleotide sequences of SEQ ID NOS: 17 and 18 and an mmoY gene fragment was amplified by PCR using a set of primers of nucleotide sequences of SEQ ID NOS: 19 and 20 as templates and a set of primers of nucleotide sequences of SEQ ID NOS: 17 and 20. A gene fragment thus amplified was ligated with pETDuet (Novagen, Cat. No. 71146-3) digested with restriction enzymes, NcoI and HindIII, using an InFusion Cloning Kit (Clontech Laboratories, Inc.) to prepare a pETDuet-mmoXY vector. Further, a region including the mmoZ and mmoD genes was amplified using an mmoZ gene fragment which was amplified by PCR using a set of primers of nucleotide sequences of SEQ ID NOS: 21 and 22 and an mmoD gene fragment which was amplified by PCR using a set of primers of nucleotide sequences of SEQ ID NOS: 23 and 24 as templates and a set of primers of nucleotide sequences of SEQ ID NOS: 21 and 24. A gene fragment thus amplified was ligated with pETDuet-mmoXY digested with restriction enzymes, NdeI and XhoI, using the InFusion Cloning Kit (Clontech Laboratories, Inc.) to prepare a pETDuet-mmoXY-ZD vector. FIG. 1B shows a vector map of the pETDuet-mmoXY-ZD vector.

A region including the mmoB and mmoC genes was amplified using an mmoB gene fragment which was amplified by PCR using a set of primers of nucleotide sequences of SEQ ID NOS: 25 and 26 and an mmoC gene fragment which was amplified by PCR using a set of primers of nucleotide sequences of SEQ ID NOS: 27 and 28 as templates and a set of primers of nucleotide sequences of SEQ ID NOS: 25 and 28. A gene fragment thus amplified was ligated with pACYCDuet (Novagen, Cat. No. 71147-3) digested with restriction enzymes, NdeI and EcoRV, using the InFusion Cloning Kit (Clontech Laboratories, Inc.) to prepare a pACYCDuet-mmoBC vector. FIG. 1C shows a vector map of the pACYCDuet-mmoBC vector.

An mmoG gene fragment which was amplified by PCR using a set of primers of nucleotide sequences of SEQ ID NOS: 29 and 30 was ligated with pACYCDuet (Novagen, Cat. No. 71147-3) digested with restriction enzymes, NcoI and HindIII, using the InFusion Cloning Kit (Clontech Laboratories, Inc.) to prepare a pACYCDuet-mmoG vector. Further, a region including the mmoB and mmoC genes was amplified using the mmoB gene fragment which was amplified by PCR using a set of primers of nucleotide sequences of SEQ ID NOS: 25 and 26 and the mmoC gene fragment which was amplified by PCR using a set of primers of nucleotide sequences of SEQ ID NOS: 27 and 28 as templates and a set of primers of nucleotide sequences of SEQ ID NOS: 25 and 28. A gene fragment thus amplified was ligated with pACYCDuet-mmoG digested with restriction enzymes, NdeI and EcoRV, using the InFusion Cloning Kit (Clontech Laboratories, Inc.) to prepare a pACYCDuet-mmoG-BC vector. FIG. 1D shows a vector map of the pACYCDuet-mmoG-BC vector.

Next, E. coli BL21 strain was introduced with each of the prepared pETDuet-mmoXY-ZD vector and pACYCDuet-mmoBC vector, pETDuet-mmoXY-ZD and pACYCDuet-mmoG-BC vector, and pET28a-mmoXYBZDC vector by a heat shock method, and then cultured on a LB plate containing 100 μg/mL of ampicillin and 35 μg/mL of chloramphenicol or 50 μg/mL of kanamycin. Strains showing ampicillin resistance and chloramphenicol or kanamycin resistance were selected. Finally, three kinds of strains selected were designated as recombinant E. coli BL21/pETDuet-mmoXY-ZD+pACYCDuet-mmoBC, BL21/pETDuet-mmoXY-ZD+pACYCDuet-mmoG-BC, and BL21/pET28a-mmoXYBZDC.

(2) Effect of Removing CHF₃ or CHCl₃ in Sample by Recombinant E. coli Expressing sMMO Gene

The sMMO gene-introduced, recombinant E. coli BL21/pETDuet-mmoXY-ZD+pACYCDuet-mmoBC, BL21/pETDuet-mmoXY-ZD+pACYCDuet-mmoG-BC, and BL21/pET28a-mmoXYBZDC prepared in section (1) were examined to determine their ability to affect removal of CHF₃ or CHCl₃ in a sample. As a control group, E. coli BL21/pETDuet+pACYCDuet or BL21/pET28a introduced with an empty vector containing no sMMO gene was used.

Each of the recombinant E. coli BL21/pETDuet-mmoXY-ZD+pACYCDuet-mmoBC, BL21/pETDuet-mmoXY-ZD+pACYCDuet-mmoG-BC, and BL21/pET28a-mmoXYBZDC was cultured in a Terrific Broth (TB) medium under stirring at 30° C. and 230 rpm. At OD₆₀₀ of about 0.5, 0.1 mM of IPTG and 0.1 mg/ml of ferric citrate, 0.1 mg/ml of ferrous sulfate, 0.1 mg/ml of ferric ammonium citrate, and 1 mM of cysteine were added thereto, followed by culturing at 25° C. and 230 rpm overnight. With respect to each recombinant E. coli, cells were harvested and suspended in an M9 medium containing 4 g/L of glucose to a cell density of OD₆₀₀ of 2.5. Each 10 ml of the cell suspensions was added to a 60 ml-serum bottle, and the bottles were sealed. The TB medium included 12 g of tryptone, 24 g of yeast extract, 5 g of glycerol, and 89 mM phosphate buffer per 1 L of distilled water. Further, the M9 medium included 6 g of Na₂HPO₄, 3 g of KH₂PO₄, 0.5 g of NaCl, and 1 g of NH₄Cl per 1 L of distilled water.

Next, in the case of CHF₃ reaction, gas-phase CHF₃ was injected through a rubber stopper of a cap of the serum bottle using a syringe to its headspace concentration of 1000 ppm. Further, in the case of CHCl₃ reaction, liquid-phase CHCl₃ was injected through the rubber stopper of the cap of the serum bottle using the syringe to its concentration of 0.02 mM in the medium. Thereafter, the serum bottle for CHF₃ reaction was incubated for 94 hours, and the serum bottle for CHCl₃ reaction was incubated for 25 hours, while stirring at 30° C. and 200 rpm. Each experiment was performed in triplicate.

After a predetermined time during incubation, 0.5 ml of the headspace gas containing no medium in the serum bottle was collected using a 1.0 ml-headspace syringe and injected into GC (Agilent 7890, Palo Alto, Calif., USA). The injected CHF₃ or CHCl₃ was separated through a CP-PoraBOND Q column (25 m length, 0.32 mm i.d., 5 um film thickness, Agilent), and changes in the CHF₃ or CHCl₃ concentration were analyzed by MSD (Agilent 5973, Palo Alto, Calif., USA). As a carrier gas, helium was used, and applied to the column at a flow rate of 1.5 ml/min. GC conditions were as follows: An inlet temperature was 250° C., an initial temperature was maintained at 40° C. for 2 minutes, and temperature was raised to 290° C. at a rate of 20° C./min. MS conditions were as follows: Ionization energy was 70 eV, an interface temperature was 280° C., an ion source temperature was 230° C., and a quadrupole temperature was 150° C.

FIG. 2 shows changes in a headspace concentration of CHF₃ when recombinant E. coli was cultured in the medium contacted with CHF₃-containing gas. In FIG. 2, 1 represents a control group, and 2 to 4 represent experiments which were performed using the recombinant E. coli BL21/pETDuet-mmoXY-ZD+pACYCDuet-mmoBC, BL21/pETDuet-mmoXY-ZD+pACYCDuet-mmoG-BC, and BL21/pET28a-mmoXYBZDC, respectively. As shown in FIG. 2, when the recombinant E. coli BL21/pETDuet-mmoXY-ZD+pACYCDuet-mmoBC was cultured for 94 hours, the headspace concentration of CHF₃ was decreased by about 10%, compared to the control group. Further, when each of the recombinant E. coli BL21/pETDuet-mmoXY-ZD+pACYCDuet-mmoG-BC and BL21/pET28a-mmoXYBZDC was cultured for 94 hours, the headspace concentration of CHF₃ was decreased by about 15%, compared to the control group.

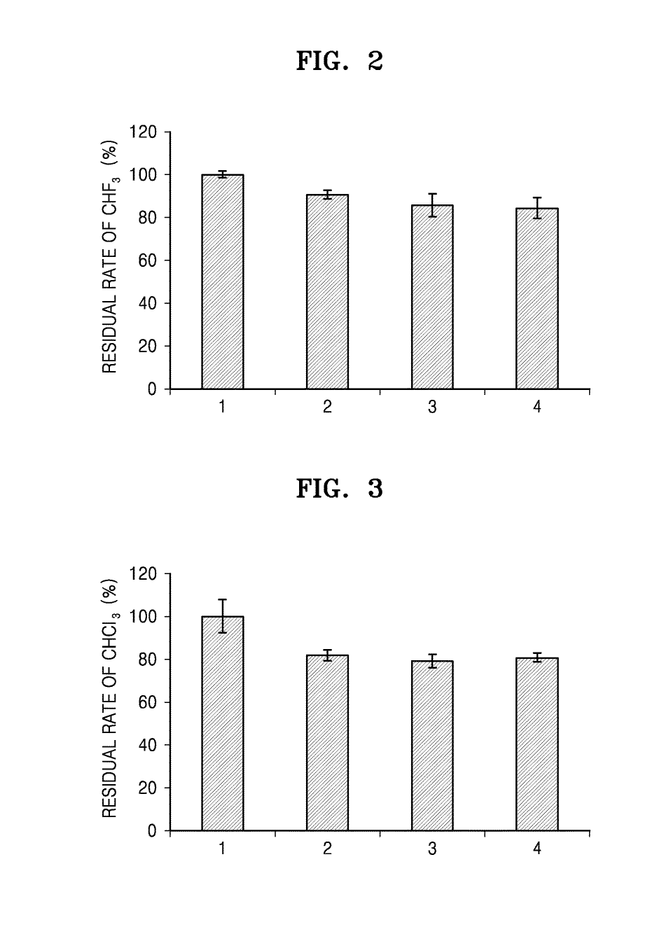

FIG. 3 shows changes in a headspace concentration of CHCl₃ when recombinant E. coli was cultured in a CHCl₃-containing medium. In FIG. 3, 1 to 4 are the same as described in FIG. 2. As shown in FIG. 3, when each of the recombinant E. coli BL21/pETDuet-mmoXY-ZD+pACYCDuet-mmoBC and BL21/pETDuet-mmoXY-ZD+pACYCDuet-mmoG-BC was cultured for 25 hours, the headspace concentration of CHCl₃ was decreased by about 20%, compared to the control group. Further, when BL21/pET28a-mmoXYBZDC was cultured for 25 hours, the headspace concentration of CHCl₃ was also decreased by the level similar thereto.

### Example 2: Effect of Removing CF₄ in Sample by Recombinant E. coli Expressing sMMO or Variant Thereof

(1) Effect of Removing CF₄ in Sample by Recombinant E. coli Expressing sMMO Gene

The sMMO gene-introduced, E. coli BL21/pET28a-mmoXYBZDC strain prepared in section 1(1) was examined to determine whether the introduced sMMO gene affects removal of CF₄ in a sample. As a control group, E. coli BL21/pET28a introduced with an empty vector containing no sMMO gene was used.

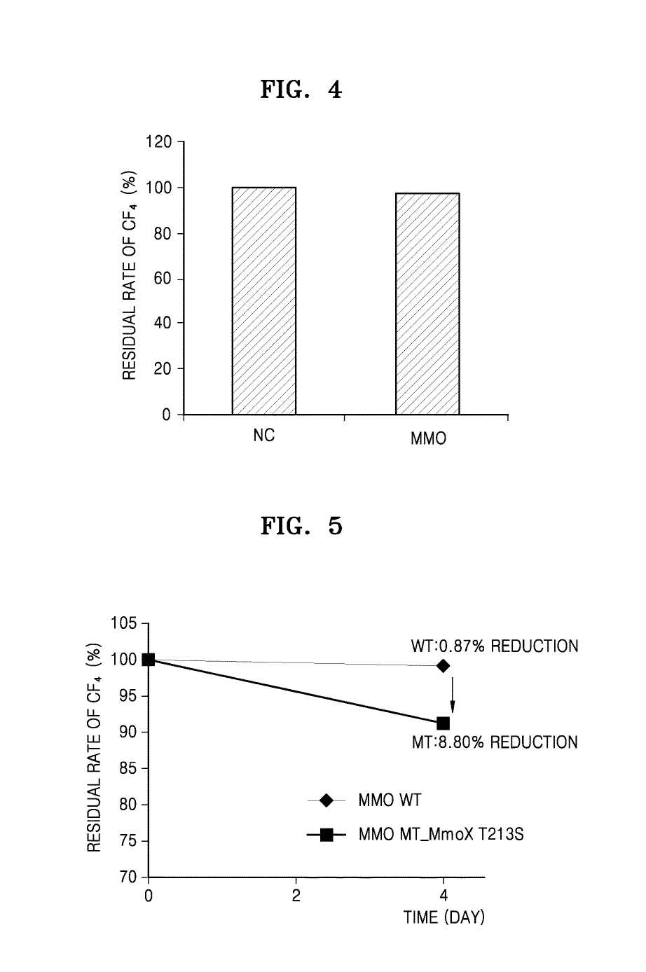

The experiment was performed in the same manner as the procedure performed for CHF₃ in Section 1(2), except that CF₄ was used instead of CHF₃ and gas-phase CF₄ was injected through a rubber stopper of a cap of the serum bottle using a syringe to its headspace concentration of 1000 ppm, and then the serum bottle was incubated for 7 days, while stirring at 30° C. and 200 rpm. The results are as shown in FIG. 4.

FIG. 4 shows changes in a headspace concentration of CF₄ over time when recombinant E. coli BL21/pET28a-mmoXYBZDC was cultured for 7 days in a medium contacted with CF₄-containing gas. In FIG. 4, NC presents a negative control group and ‘MMO’ represents the experiment performed by using E. coli BL21/pET28a-mmoXYBZDC. As shown in FIG. 4, when the E. coli BL21/pET28a-mmoXYBZDC was cultured for 7 days, the headspace concentration of CF₄ was decreased by about 3.42%, compared to the control group.

(2) Recombinant E. coli Expressing Mutant sMMO Gene and its Effect of Removing CF₄ in Sample

In this section, mutants were prepared in order to improve the activity of removing fluorinated methane in a sample by the sMMO complex including MmoX. Threonine (hereinafter, referred to as “T213”) at position 213 of the amino acid sequence of SEQ ID NO: 1 was replaced by other 19 natural amino acids (hereinafter, referred to as “T213”. Here, X represents 19 natural amino acids other than threonine), and each of the genes encoding the mutants was introduced into E. coli, and their activity of removing CF₄ in a sample was examined. MmoX is a factor constituting hydroxylase domain including a binuclear iron center.

(2.1) Preparation of 19 Mutants

Preparation of the T213X mutants of SEQ ID NO: 1 was performed using a QuikChange II Site-Directed Mutagenesis Kit (Agilent Technology, USA). Site-directed mutagenesis using the kit was performed using PfuUlta high-fidelity (HF) DNA polymerase for mutagenic primer-directed replication of both plasmid strands with the highest fidelity. The basic procedure utilized a supercoiled double-stranded DNA (dsDNA) vector with an insert of interest and two synthetic oligonucleotide primers, both containing the desired mutation. The oligonucleotide primers, each complementary to opposite strands of the vector, were extended during temperature cycling by PfuUltra HF DNA polymerase, without primer displacement. Extension of the oligonucleotide primers generated a mutated plasmid containing staggered nicks. Following temperature cycling, the product was treated with Dpn I. The Dpn I endonuclease (target sequence: 5′-Gm⁶ATC-3′) was specific for methylated and hemimethylated DNA and was used to digest the parental DNA template and to select for mutation-containing synthesized DNA. The nicked vector DNA incorporating the desired mutations was then transformed into XL1-Blue supercompetent cells. Of respective primer sets used to induce T213X mutation, primer sets regarding to the increased activity of removing fluorinated methane in a sample, compared to that of the wild-type E. coli, are given in the following Table 2.

TABLE 2

| No. | Mutation type | Primer sequence |
|---|---|---|
| 1 | T213Y | SEQ ID NOS: 32 and 33 |
| 2 | T213S | SEQ ID NOS: 34 and 35 |
| 3 | T213K | SEQ ID NOS: 36 and 37 |
| 4 | T213H | SEQ ID NOS: 38 and 39 |
| 5 | T213E | SEQ ID NOS: 40 and 41 |

In detail, PCR was performed using the pET28a-mmoXYBZDC vector prepared in Example (1) as a template and each of the primer sets described in Table 1 as a primer and PfuUlta HF DNA polymerase to obtain mutated vectors containing staggered nicks. These vector products were treated with DpnI to select mutation-containing synthesized DNAs. The vectors DNA incorporating nicks including the desired mutations were then transformed into XL1-Blue supercompetent cells to clone a pET28a-mmoXYBZDCmt vector.

Lastly, the cloned pET28a-mmoXYBZDCmt vector and pET28a-mmoXYBZDCwt vector were introduced into E. coli BL21 strain in the same manner as in Example (1), and a finally selected strain was designated as recombinant E. coli BL21/pET28a-mmoXYBZDCmt.

(2.2) Effect of Removing CF₄ in Sample by Recombinant E. coli BL21/pET28a-mmoXYBZDCmt

In this section, it was examined whether the mutant MmoX gene-introduced, E. coli BL21/pET28a-mmoXYBZDCmt prepared in section (2.1) affects removal of CF₄ in a sample.

The experiment was performed in the same manner as the procedure performed for CHF₃ in Section 1(2), except that CF₄ was used instead of CHF₃ and gas-phase CF₄ was injected through a rubber stopper of a cap of the serum bottle using a syringe to its headspace concentration of 1000 ppm, and then the serum bottle was incubated for 6 days, while stirring at 30° C. and 230 rpm. The results are as shown in Table 3.

TABLE 3

| | | Residual amount of CF₄ | Reduction amount of CF₄ |
|---|---|---|---|
| | Mutation | (Percentage relative to | (Percentage relative to |
| NO. | type | control group) | control group) |
| 1 | T213Y | 93.08 | 6.92 |
| 2 | T213S | 92.12 | 7.88 |
| 3 | T213K | 92.41 | 7.59 |
| 4 | T213H | 93.82 | 6.18 |
| 5 | T213E | 90.26 | 9.74 |
| 6 | T213* | 96.58 | 3.42 |

In Table 3, the control group represents E. coli introduced with the pET28a vector instead of the pET28a-mmoXYBZDCmt vector, and T213* represents E. coli containing wild-type MmoX.

Further, in this section, the experiment was performed in the same manner as the procedure performed for CHF₃ in Section 1(2), except that 20 mL of mutant MmoX-introduced E. coli BL21/pET28a-mmoXYBZDCmt (OD₆₀₀=3.0) prepared in Section (2.1) was injected to a 175-mL flask, CF₄ was used instead of CHF₃, and gas-phase CF₄ was injected through a rubber stopper of a cap of the serum bottle using a syringe to its headspace concentration of 1000 ppm, and then the serum bottle was incubated for 4 days, while stirring at 30° C. and 230 rpm. A residual amount of CF₄ over time, that is, a remaining percentage (%) of CF₄ was examined. The results are shown in FIG. 5.

FIG. 5 shows changes of CF₄ in a sample over time by E. coli BL21/pET28a-mmoXYBZDCmt introduced with the mutant MmoX gene. As shown in FIG. 5, when the recombinant E. coli MMO MT_MmoX T213S mutant gene-containing strain was cultured for 4 days, the CF₄ level was decreased by about 8.80%, compared to the control group. In contrast, the wild-type strain decreased the CF₄ level by about 0.87%, compared to the control group.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising an exogenous gene encoding a soluble methane monooxygenase (sMMO) protein or a variant thereof, wherein the variant is a MmoX variant having an amino acid alteration at an amino acid residue corresponding to the threonine at position 213 of SEQ ID NO: 1 and having an activity belonging to EC 1.14.13.25.

2. The recombinant microorganism of claim 1, wherein the amino acid alteration is replacement of the amino acid residue corresponding to the threonine at position 213 of SEQ ID NO: 1 with Y, S, K, H, or E.

3. The recombinant microorganism of claim 1, wherein the sMMO protein comprises MmoX or a variant thereof, MmoY, MmoZ, MmoB, MmoC, and MmoD; and the MmoX or variant thereof, MmoY, MmoZ, MmoB, MmoC, and MmoD have 95% or higher sequence identity to amino acid sequences of SEQ ID NOS: 1, 3, 5, 7, 9, and 11, respectively.

4. The recombinant microorganism of claim 1, wherein the gene comprises a polynucleotide having a nucleotide sequence of SEQ ID NO: 2 or a polynucleotide encoding the MmoX variant having an amino acid alteration at an amino acid residue corresponding to the position T213 in MmoX of the amino acid sequence of SEQ ID NO: 1, a polynucleotide having a nucleotide sequence of SEQ ID NO: 4, a polynucleotide having a nucleotide sequence of SEQ ID NO: 6, a polynucleotide having a nucleotide sequence of SEQ ID NO: 8, a polynucleotide having a nucleotide sequence of SEQ ID NO: 10, and a polynucleotide having a nucleotide sequence of SEQ ID NO: 12.

5. The recombinant microorganism of claim 1, wherein the gene comprises the nucleotide sequence of SEQ ID NO: 31.

6. The recombinant microorganism of claim 1, wherein the microorganism further comprises an exogenous gene encoding MmoG, and MmoG has 95% or higher sequence identity to an amino acid sequence of SEQ ID NO: 13.

7. A method of reducing a concentration of fluorinated methane in a sample, the method comprising contacting an sMMO or a variant thereof with a sample comprising fluorinated methane represented by CHₙF₄₋ₙ (n is an integer of 0 to 3) to reduce the concentration of fluorinated methane in the sample, wherein the variant comprises a MmoX variant having an amino acid alteration at an amino acid residue corresponding to position T213 in MmoX of the amino acid sequence of SEQ ID NO: 1 and having an activity belonging to EC 1.14.13.25.

8. The method of claim 7, wherein the amino acid alteration is replacement of the amino acid residue corresponding to position T213 of SEQ ID NO: 1 with Y, S, K, H, or E.

9. The method of claim 7, wherein the sMMO protein comprises MmoX or a variant thereof, MmoY, MmoZ, MmoB, MmoC, and MmoD, and MmoX or the variant thereof, MmoY, MmoZ, MmoB, MmoC, and MmoD have 95% or higher sequence identity to amino acid sequences of SEQ ID NOS: 1, 3, 5, 7, 9, and 11, respectively.

10. The method of claim 7, wherein the sMMO protein or the variant thereof is in the form of a recombinant microorganism comprising the expressed protein or variant thereof.

11. The method of claim 10, wherein contacting the sMMO with the sample comprises culturing the recombinant microorganism in the presence of fluorinated methane.

12. A MmoX variant having an amino acid alteration at an amino acid residue corresponding to the position T213 in MmoX of an amino acid sequence of SEQ ID NO: 1 and having an activity belonging to EC 1.14.13.25, or a sMMO protein complex comprising the MmoX variant.

13. A polynucleotide encoding a MmoX variant having an amino acid alteration at an amino acid residue corresponding to the position T213 in MmoX of an amino acid sequence of SEQ ID NO: 1 and having an activity belonging to EC 1.14.13.25 or a sMMO protein complex comprising the MmoX variant.


SEQUENCE LISTING (46 sequences)

SEQ ID NO: 1 - protein, 527 aa, Methylococcus capsulatus
  misc_feature (1)..(527): (Bath)
MALSTATKAATDALAANRAPTSVNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWNRWVYEDWGGIWIGRLGKYGVESPRSLKDAKQDAYWAHHDLYLLAYALWPTGFFRLALPDQEEMEWFEANYPGWYDHYGKIYEEWRARGCEDPSSGFIPLMWFIENNHPIYIDRVSQVPFCPSLAKGASTLRVHEYNGQMHTFSDQWGERMWLAEPERYECQNIFEQYEGRELSEVIAELHGLRSDGKTLIAQPHVRGDKLWTLDDIKRLNCVFKNPVKAFN

SEQ ID NO: 2 - DNA, 1584 bp, Methylococcus capsulatus
  misc_feature (1)..(1584): (Bath)
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 3 - protein, 389 aa, Methylococcus capsulatus
  misc_feature (1)..(389): (Bath)
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASRIDFKADRDQIVKAVLAGLK

SEQ ID NO: 4 - DNA, 1170 bp, Methylococcus capsulatus
  misc_feature (1)..(1170): (Bath)
atgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataa

SEQ ID NO: 5 - protein, 170 aa, Methylococcus capsulatus
  misc_feature (1)..(170): (Bath)
MAKLGIHSNDTRDAWVNKIAQLNTLEKAAEMLKQFRMDHTTPFRNSYELDNDYLWIEAKLEEKVAVLKARAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 6 - DNA, 513 bp, Methylococcus capsulatus
  misc_feature (1)..(513): (Bath)
atggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactga

SEQ ID NO: 7 - protein, 141 aa, Methylococcus capsulatus
  misc_feature (1)..(141): (Bath)
MSVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLEDYHA

SEQ ID NO: 8 - DNA, 426 bp, Methylococcus capsulatus
  misc_feature (1)..(426): (Bath)
atgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactatcacgcttaa

SEQ ID NO: 9 - protein, 348 aa, Methylococcus capsulatus
  misc_feature (1)..(348): (Bath)
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 10 - DNA, 1047 bp, Methylococcus capsulatus
  misc_feature (1)..(1047): (Bath)
atgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctaa

SEQ ID NO: 11 - protein, 103 aa, Methylococcus capsulatus
  misc_feature (1)..(103): (Bath)
MVESAFQPFSGDADEWFEEPRPQAGFFPSADWHLLKRDETYAAYAKDLDFMWRWVIVREERIVQEGCSISLESSIRAVTHVLNYFGMTEQRAPAEDRTGGVQH

SEQ ID NO: 12 - DNA, 312 bp, Methylococcus capsulatus
  misc_feature (1)..(312): (Bath)
atggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattga

SEQ ID NO: 13 - protein, 559 aa, Methylococcus capsulatus
  misc_feature (1)..(559): (Bath)
MAKEVVYRGSARQRMMQGIEILARAAIPTLGATGPSVMIQHRADGLPPISTRDGVTVANSIVLKDRVANLGARLLRDVAGTMSREAGDGTTTAIVLARHIAREMFKSLAVGADPIALKRGIDRAVARVSEDIGARAWRGDKESVILGVAAVATKGEPGVGRLLLEALDAVGVHGAVSIELGQRREDLLDVVDGYRWEKGYLSPYFVTDRARELAELEDVYLLMTDREVVDFIDLVPLLEAVTEAGGSLLIAADRVHEKALAGLLLNHVRGVFKAVAVTAPGFGDKRPNRLLDLAALTGGRAVLEAQGDRLDRVTLADLGRVRRAVVSADDTALLGIPGTEASRARLEGLRLEAEQYRALKPGQGSATGRLHELEEIEARIVGLSGKSAVYRVGGVTDVEMKERMVRIENAYRSVVSALEEGVLPGGGVGFLGSMPVLAELEARDADEARGIGIVRSALTEPLRIIGENSGLSGEAVVAKVMDHANPGWGYDQESGSFCDLHARGIWDAAKVLRLALEKAASVAGTFLTTEAVVLEIPDTDAFAGFSAEWAAATREDPRV

SEQ ID NO: 14 - DNA, 1680 bp, Methylococcus capsulatus
  misc_feature (1)..(1680): (Bath)
atggcaaaggaagtggtttacagggggagtgcgcggcagcgcatgatgcaaggcatcgagatactcgcgcgggcggcgataccgacgctgggagccaccggccccagcgtcatgatccagcaccgcgccgatggcctgccccccatttcgacgcgggacggcgtcacggtggctaactccatcgtactcaaggaccgtgtcgcgaatctcggtgcccggctgctgcgggacgtcgccggcaccatgtcccgcgaagcaggggatggcaccaccaccgccatcgtgctggcccgccatatcgcccgggagatgttcaagagcctcgccgtcggtgccgatcccatcgctctcaagcgtggtatcgaccgtgccgtcgcccgcgtgagcgaggacatcggggctcgggcctggcgcggcgacaaggaatcggtcatcctgggggtggccgcggtggcgaccaagggcgagccgggcgtgggccggctgctgctggaggcgctggacgcggtcggcgtccatggcgccgtgtcgatcgaactggggcagcggcgcgaggacctgctcgacgtggtcgacgggtatcgttgggaaaaaggttatctgtcgccctattttgtgaccgatcgggctcgcgagctggccgaactcgaagacgtctacctcttgatgaccgatcgggaggtggtcgatttcatcgatttggtacccctgctggaggcggtgaccgaggctggtggcagcctcctgatcgccgccgaccgtgtccacgagaaggcactggccggccttttgctcaatcacgttcgcggcgtcttcaaggccgtcgcggtcaccgcgcccgggttcggcgacaagcggccgaaccgccttttggatctggcggcgttgaccggtgggcgggcggtcctggaagcccagggcgaccgattggaccgggtcacgctggccgacctggggcgggtgcggcgggcggtcgtcagcgctgacgacaccgcgctgctcggcataccgggcaccgaagcctcccgggcccgcttggagggtttgcgcctggaagcggagcagtaccgggcgctcaagcccggtcagggatcggcgacggggcgcttgcacgagctcgaggaaatcgaggcccggatcgtcggtctgagcggcaagtccgcggtctaccgcgtgggcggcgtgaccgacgtggagatgaaggagcggatggtacggatcgaaaatgcctaccgctcggtggtgtctgcactggaggagggggtgttgcccggcggcggtgtcgggtttctgggcagcatgcccgttttggccgagctggaagcgcgcgatgccgacgaagcacgcggcatcggcatcgtccgttccgcgctgacggagcccctccggatcatcggagaaaattcgggactgtcaggggaggccgtcgtcgccaaggtcatggatcacgccaatcccggttggggttacgatcaggaaagcggaagtttctgcgacctccacgccaggggcatttgggatgccgccaaggtgctcaggctggccctggaaaaagccgcgtcggtggccggcacgtttctcaccaccgaagccgtggtactggagattccggacactgacgctttcgccggtttcagtgcggagtgggccgccgcgacccgggaggatccgcgggtctaa

SEQ ID NO: 15 - DNA, 36 bp, Artificial Sequence
  primer
aagaaggagatataccatggcacttagcaccgcaac

SEQ ID NO: 16 - DNA, 40 bp, Artificial Sequence
  primer
gtggtggtggtggtgctcgattaggccgccccggacggca

SEQ ID NO: 17 - DNA, 36 bp, Artificial Sequence; identical to SEQ ID NO: 15
  primer

SEQ ID NO: 18 - DNA, 39 bp, Artificial Sequence
  primer
gaattctgtttcctgtgtgattaattgaatgccttcacc

SEQ ID NO: 19 - DNA, 39 bp, Artificial Sequence
  primer
tcacacaggaaacagaattcatgagcatgttaggagaaa

SEQ ID NO: 20 - DNA, 38 bp, Artificial Sequence
  primer
cattatgcggccgcaagctttatttcaatcctgccaga

SEQ ID NO: 21 - DNA, 36 bp, Artificial Sequence
  primer
aagaaggagatatacatatggcgaaactgggtatac

SEQ ID NO: 22 - DNA, 39 bp, Artificial Sequence
  primer
gaattctgtttcctgtgtgattagtgcggcgactgcaga

SEQ ID NO: 23 - DNA, 39 bp, Artificial Sequence
  primer
tcacacaggaaacagaattcatggtcgaatcggcatttc

SEQ ID NO: 24 - DNA, 40 bp, Artificial Sequence
  primer
gtttctttaccagactcgattaatgttgaactccgccggt

SEQ ID NO: 25 - DNA, 36 bp, Artificial Sequence
  primer
aagaaggagatatacatatgagcgtaaacagcaacg

SEQ ID NO: 26 - DNA, 39 bp, Artificial Sequence
  primer
atgtatatctccttcttatattaagcgtgatagtcttcg

SEQ ID NO: 27 - DNA, 39 bp, Artificial Sequence
  primer
tataagaaggagatatacatatgcagcgagttcacacta

SEQ ID NO: 28 - DNA, 39 bp, Artificial Sequence
  primer
gatcgcgtggccggccgatttaggccgccccggacggca

SEQ ID NO: 29 - DNA, 36 bp, Artificial Sequence
  primer
taataaggagatataccatggcaaaggaagtggttt

SEQ ID NO: 30 - DNA, 38 bp, Artificial Sequence
  primer
cattatgcggccgcaagctttagacccgcggatcctcc

SEQ ID NO: 31 - DNA, 5324 bp, Methylococcus capsulatus
  misc_feature (1)..(5324): (Bath)
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactatcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggccggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctaa

SEQ ID NO: 32 - DNA, 39 bp, Artificial Sequence
  Synthetic Forward primer for T213Y
gtgggtgaggcctgcttctataatccgctgatcgtcgca

SEQ ID NO: 33 - DNA, 39 bp, Artificial Sequence
  Synthetic Reverse primer for T213Y
tgcgacgatcagcggattatagaagcaggcctcacccac

SEQ ID NO: 34 - DNA, 39 bp, Artificial Sequence
  Synthetic Forward primer for T213S
gtgggtgaggcctgcttcagcaatccgctgatcgtcgca

SEQ ID NO: 35 - DNA, 39 bp, Artificial Sequence
  Synthetic Reverse primer for T213S
tgcgacgatcagcggattgctgaagcaggcctcacccac

SEQ ID NO: 36 - DNA, 39 bp, Artificial Sequence
  Synthetic Forward primer for T213K
gtgggtgaggcctgcttcaaaaatccgctgatcgtcgca

SEQ ID NO: 37 - DNA, 39 bp, Artificial Sequence
  Synthetic Reverse primer for T213K
tgcgacgatcagcggatttttgaagcaggcctcacccac

SEQ ID NO: 38 - DNA, 39 bp, Artificial Sequence
  Synthetic Forward primer for T213H
gtgggtgaggcctgcttccataatccgctgatcgtcgca

SEQ ID NO: 39 - DNA, 39 bp, Artificial Sequence
  Synthetic Reverse primer for T213H
tgcgacgatcagcggattatggaagcaggcctcacccac

SEQ ID NO: 40 - DNA, 39 bp, Artificial Sequence
  Synthetic Forward primer for T213E
gtgggtgaggcctgcttcgaaaatccgctgatcgtcgca

SEQ ID NO: 41 - DNA, 39 bp, Artificial Sequence
  Synthetic Reverse primer for T213E
tgcgacgatcagcggattttcgaagcaggcctcacccac

SEQ ID NO: 42 - DNA, 1584 bp, Artificial Sequence
  Synthetic MmoX T213Y
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttctataatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 43 - DNA, 1584 bp, Artificial Sequence
  Synthetic MmoX T213S
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcagcaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 44 - DNA, 1584 bp, Artificial Sequence
  Synthetic MmoX T213K
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaaaaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 45 - DNA, 1584 bp, Artificial Sequence
  Synthetic MmoX T213H
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttccataatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 46 - DNA, 1584 bp, Artificial Sequence
  Synthetic MmoX T213E
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcgaaaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga